(19) United States
(12) Patent Application Publication
(10) Pub. No.: US 2021/0005300 A1
(43) Pub. Date: Jan. 7, 2021

(54) CUSTOMIZABLE COMMUNICATION PLATFORM BUILDER

(71) Applicant: CAREMINDR Corporation, Los Gatos, CA (US)

(72) Inventors: Harry Raymond Soza, San Jose, CA (US); Irina Yermilov, Los Angeles, CA (US)

(21) Appl. No.: 16/919,030

(22) Filed: Jul. 1, 2020

Related U.S. Application Data
(60) Provisional application No. 62/869,483, filed on Jul. 1, 2019.
(60) Provisional application No. 62/869,497, filed on Jul. 1, 2019.

(51) Int. Cl. G16H 20/00; G16H 80/00; G16H 40/67; G16H 10/60; G06F 3/0486

(52) U.S. Cl. CPC G16H 20/00; G16H 80/00; G06F 3/0486; G16H 10/60; G16H 40/67

(57) ABSTRACT

Processing patient information and other treatment plan information may require access to a patient profile and other third parties involved in the patient treatment plan. One example method includes displaying a treatment plan table via a cloud based server, for a patient having a set of treatment information including at least one selectable parameter block, wherein the at least one selectable parameter block has a recordation input type, a recordation input frequency, a communication schedule, at least one guardrail value, at least one flag value, and a message text query, receiving a treatment plan identifier from a healthcare provider server, populating the treatment plan table with the at least one selectable parameter block and the treatment plan identifier, creating a treatment plan based on the populated treatment plan table and sending the treatment plan to a patient device.

### TECHNICAL FIELD

This application relates to a customizable communication platform and more particularly to providing customized communication to a user device.

### BACKGROUND

Conventionally, the approach to providing users with ongoing communications regarding a plan or other repetitive course of action may leave the majority of the work to the user. The smartphone and other personal computing devices are not being properly utilized when offering users with options for maintaining a course of treatment or a set of goals. The lack of action taken by the professional service provider and/or the user can lead to personal health problems and lost revenue for providers, insurers, etc., as well as the users.

### SUMMARY

An example embodiment of the present application provides a method that includes at least one of displaying a treatment plan table via a cloud based server, for a patient having a set of treatment information including at least one selectable parameter block, wherein the at least one selectable parameter block has a recordation input type, a recordation input frequency, a communication schedule, at least one guardrail value, at least one flag value, and a message text query, receiving a treatment plan identifier from a healthcare provider server, populating the treatment plan table with the at least one selectable parameter block and the treatment plan identifier, creating a treatment plan based on the populated treatment plan table and sending the treatment plan to a patient device.

Another example embodiment of the present application provides a non-transitory computer readable medium comprising instructions that, when read by a processor, cause the processor to perform at least one of displaying a treatment plan table via a cloud based server, for a patient having a set of treatment information including at least one selectable parameter block, wherein the at least one selectable parameter block has a recordation input type, a recordation input frequency, a communication schedule, at least one guardrail value, at least one flag value, and a message text query, receiving a treatment plan identifier from a healthcare provider server, populating the treatment plan table with the at least one selectable parameter block and the treatment plan identifier, creating a treatment plan based on the populated treatment plan table and sending the treatment plan to a patient device.

A further example embodiment of the present application provides a system, comprising at least one cloud-based processor, and at least one memory electrically coupled to the at least one cloud-based processor and storing an application, wherein the at least one cloud-based processor performs at least one operation to display a treatment plan table via a cloud based server, for a patient having a set of treatment information including at least one selectable parameter block, wherein the at least one selectable parameter block has a recordation input type, a recordation input frequency, a communication schedule, at least one guardrail value, at least one flag value, and a message text query, receive a treatment plan identifier from a healthcare provider server, populate the treatment plan table with the at least one selectable parameter block and the treatment plan identifier, create a treatment plan based on the populated treatment plan table and send the treatment plan to a patient device.

### BRIEF DESCRIPTION OF THE DRAWINGS

FIG. 1 illustrates an example of the integrated application platform according to example embodiments.

FIG. 2 illustrates a network configuration of the third party participants of the integrated application according to example embodiments.

FIG. 3 illustrates a user smartphone interface of an example treatment plan according to example embodiments.

FIG. 4A illustrates an example setup configuration for a new course of treatment according to example embodiments.

FIG. 4B illustrates an example database entry for the new course of treatment according to example embodiments.

FIG. 4C illustrates a flow diagram configuration for the new course of treatment according to example embodiments.

FIG. 4D illustrates an example list of messages for the ongoing course of treatment according to example embodiments.

FIG. 4E illustrates an example setup configuration for various courses of treatment according to example embodiments.

FIG. 4F illustrates an example set of details of an ongoing course of treatment according to example embodiments.

FIG. 4G illustrates an example network configuration of the various third parties involved in the application operation and compliance according to example embodiments.

FIG. 5 illustrates a logic module configured to process the input and output parameters of the application according to example embodiments.

FIG. 6 illustrates an example network entity device configured to store instructions, software, and corresponding hardware for executing the same, according to example embodiments of the present application.

FIG. 7 illustrates a first method, according to example embodiments of the present application.

FIG. 8 illustrates a communication platform builder screen layout, according to example embodiments of the present application.

FIG. 9 illustrates a second method, according to example embodiments of the present application.

### DETAILED DESCRIPTION

It will be readily understood that the components of the present application, as generally described and illustrated in the figures herein, may be arranged and designed in a wide variety of different configurations. Thus, the following detailed description of the embodiments of a method, apparatus, and system, as represented in the attached figures, is not intended to limit the scope of the application as claimed, but is merely representative of selected embodiments of the application.

The features, structures, or characteristics of the application described throughout this specification may be combined in any suitable manner in one or more embodiments. For example, the usage of the phrases “example embodiments”, “some embodiments”, or other similar language, throughout this specification refers to the fact that a particular feature, structure, or characteristic described in connection with the embodiment may be included in at least one embodiment of the present application. Thus, appearances of the phrases “example embodiments”, “in some embodiments”, “in other embodiments”, or other similar language, throughout this specification do not necessarily all refer to the same group of embodiments, and the described features, structures, or characteristics may be combined in any suitable manner in one or more embodiments.

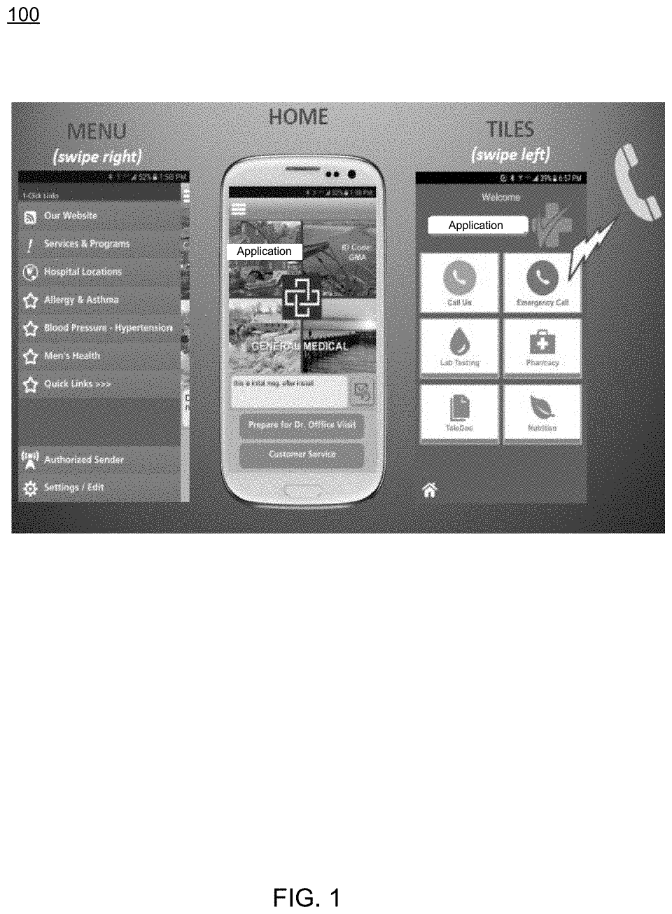

FIG. 1 illustrates an example of the integrated application platform according to example embodiments. Referring to FIG. 1, the configuration 100 includes a menu user interface, a home user interface and a set of option tiles for accessing third party resources, such as test results, emergency concerns, pharmacy information, etc. The tiles may initially be provided by the cloud system and may serve as a conduit of previous learning pertaining to a specific user's condition, treatment plan and schedule. The tiles may be added to by healthcare professionals, in which case the cloud system will include the additional information to perform additional linkages between conditions, treatment plans, drugs, timings and the like. The tiles have within them not just the specific item, but the interlinkages that make insight into specific outcomes much more insightful and make the computer outputs much more targeted.

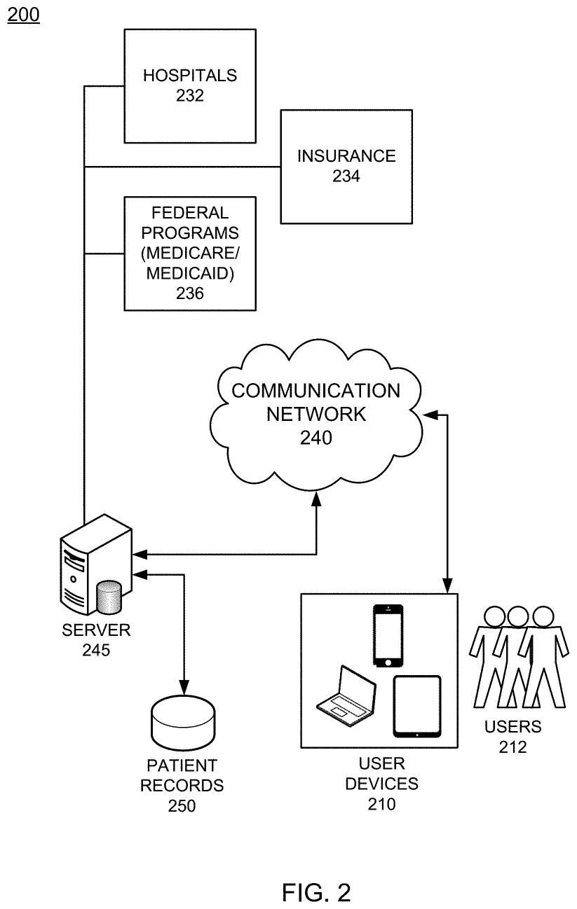

FIG. 2 illustrates a network configuration of the third party participants of the integrated application according to example embodiments. Referring to FIG. 2, the network includes a central server 245 with patient records 250. The information needed to provide treatment plans and other integrated services may require access to hospital and other provider services 232, insurance company information 234, drug providers, federal program administrators 236, etc. The information may be incorporated into any treatment plan or other integrated service model accessed by a user device 210 operated by a user 212. The servers and third party modules may operate on-site or in a cloud network managed by the providers.

Examples of treatment plans and other objectives may include a care management service for assessment of patient medical needs. The system and application may ensure timely receipt of all recommended treatment actions, drugs, third party services over a designated period of time. Also, referrals to other providers and additional services may provide emergency visits, discharge instructions, nursing facility operations, and home health care functions. In operation, the procedure may begin with the medical treatment provider creating a treatment plan or ‘journey’ for each patient. Each journey is generally for a single chronic condition or objective. One patient may have multiple journeys integrated into a single application. Also, the journeys may originate from various different providers and service entities.

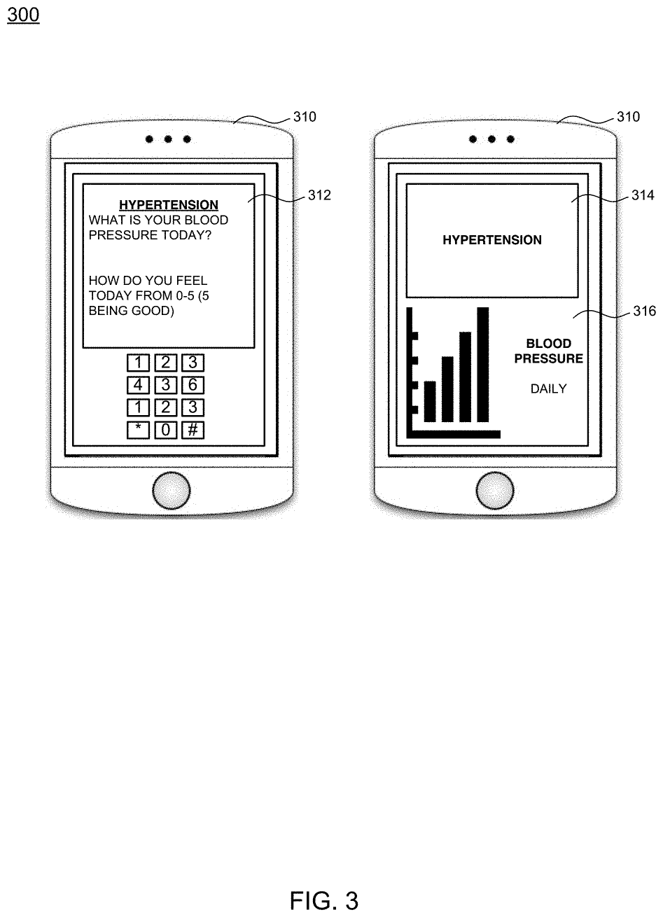

FIG. 3 illustrates a user smartphone interface of an example treatment plan according to example embodiments. Referring to FIG. 3, the journey for “hypertension” may have been created by a patient doctor and may include an interface 300 with a smartphone device 310 and a screen option configuration providing a set of questions 312, information about the treatment, reminders and other functions. The example in FIG. 3 provides for a set of questions 312 and a journey topic 314 along with a graph of blood pressure records 316 as measured over time from various interactions. The system has the capability to connect the application on the patient device to a machine, via a wireless connection such as Bluetooth, such that it may provide medical information directly to the application. Utilizing this capability, the patent device may receive a current reading such as temperature, blood pressure, heart rate, weight, etc., and have that information dynamically provided to the application which may use that information to provide immediate feedback and recommendations.

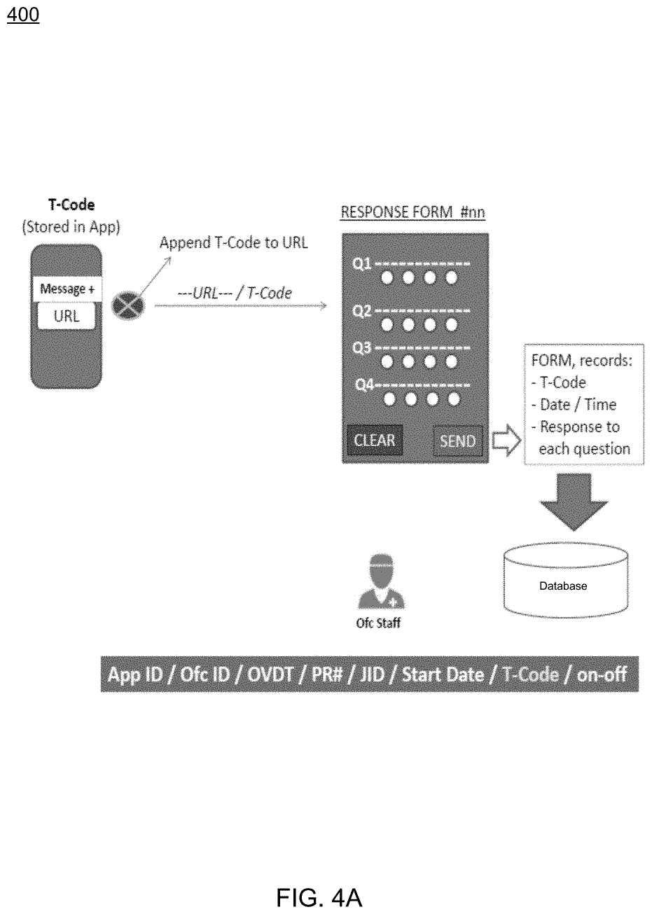

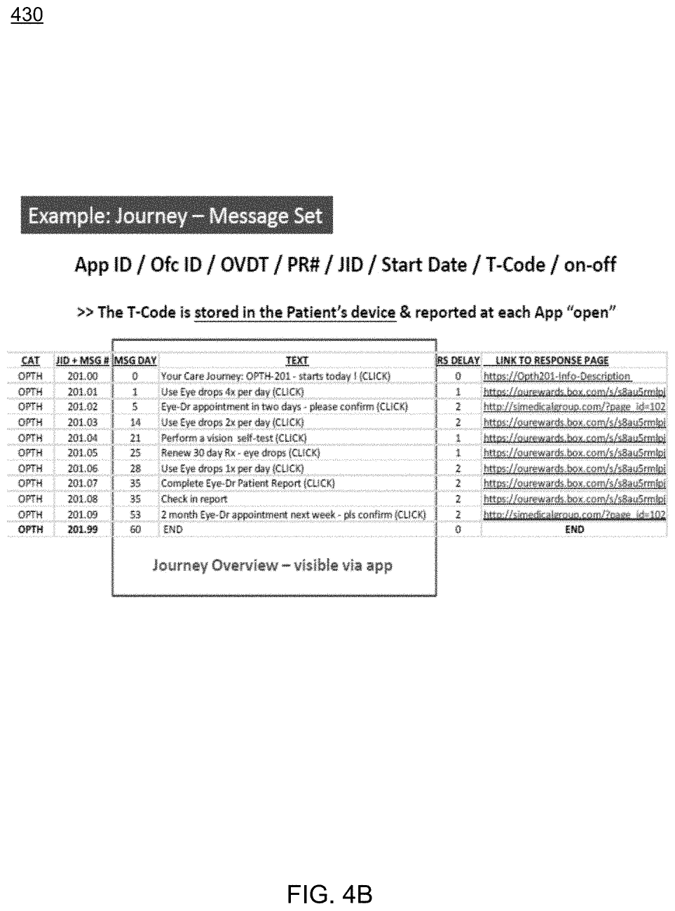

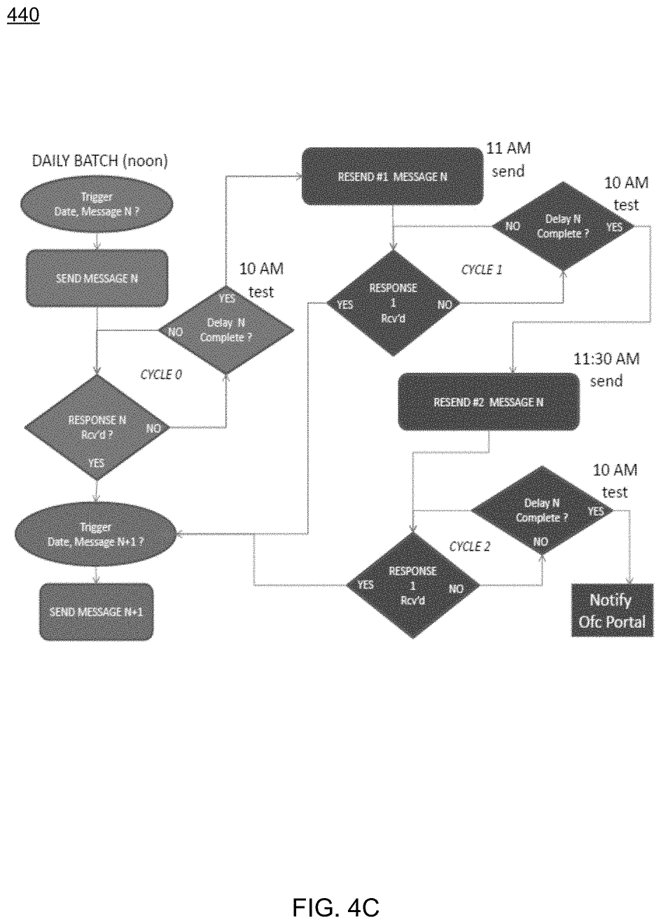

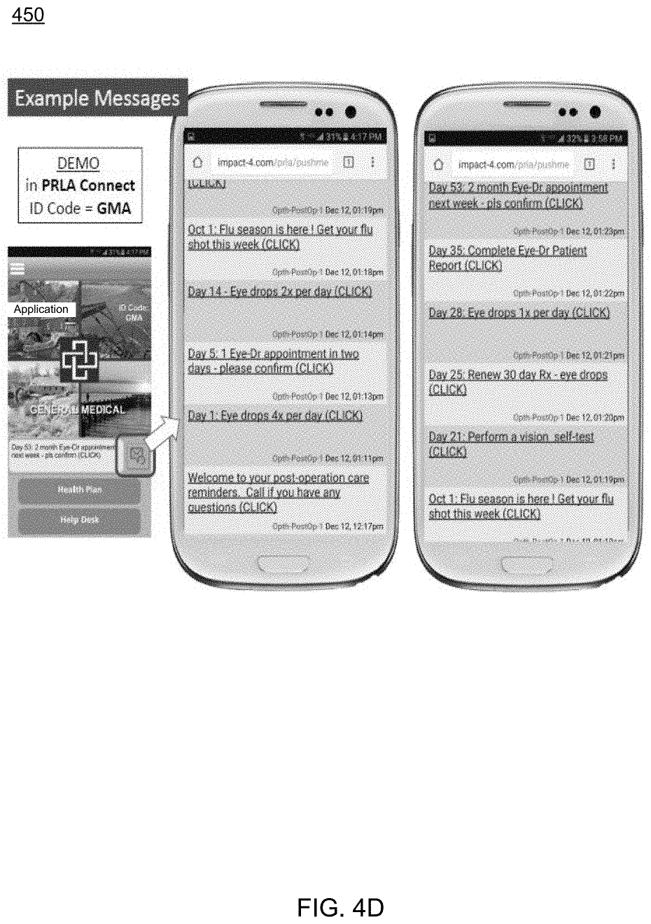

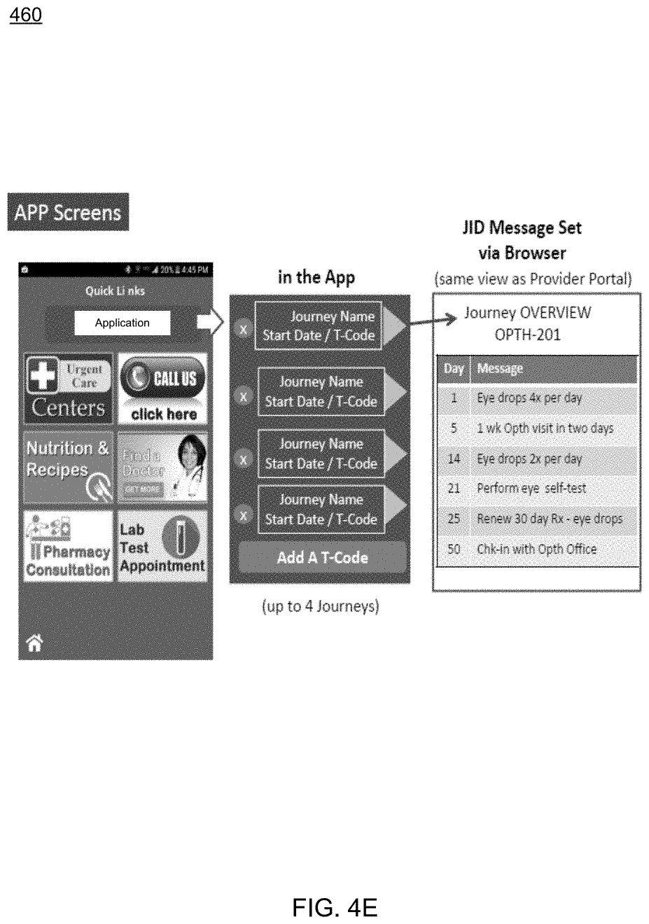

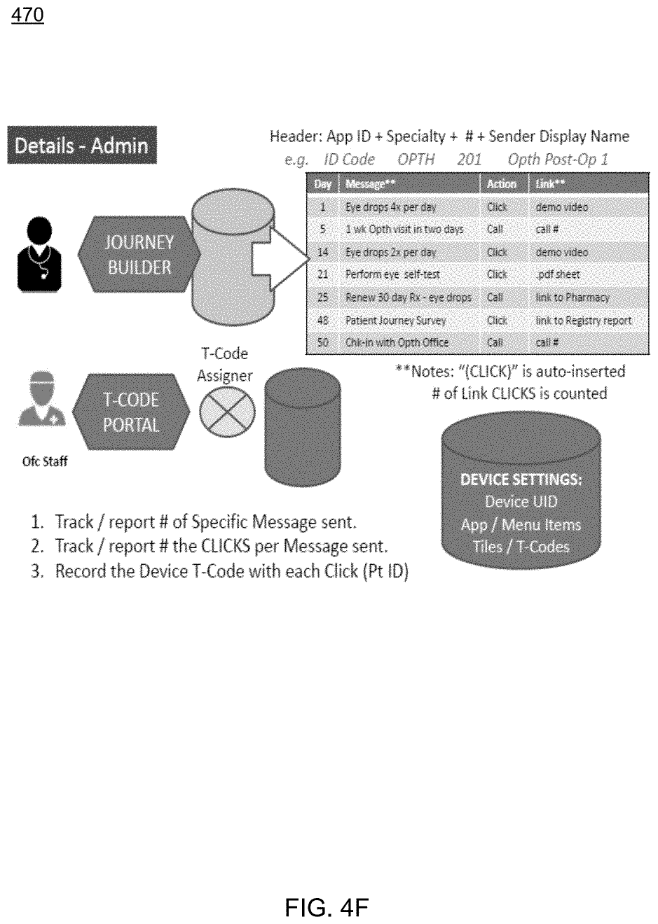

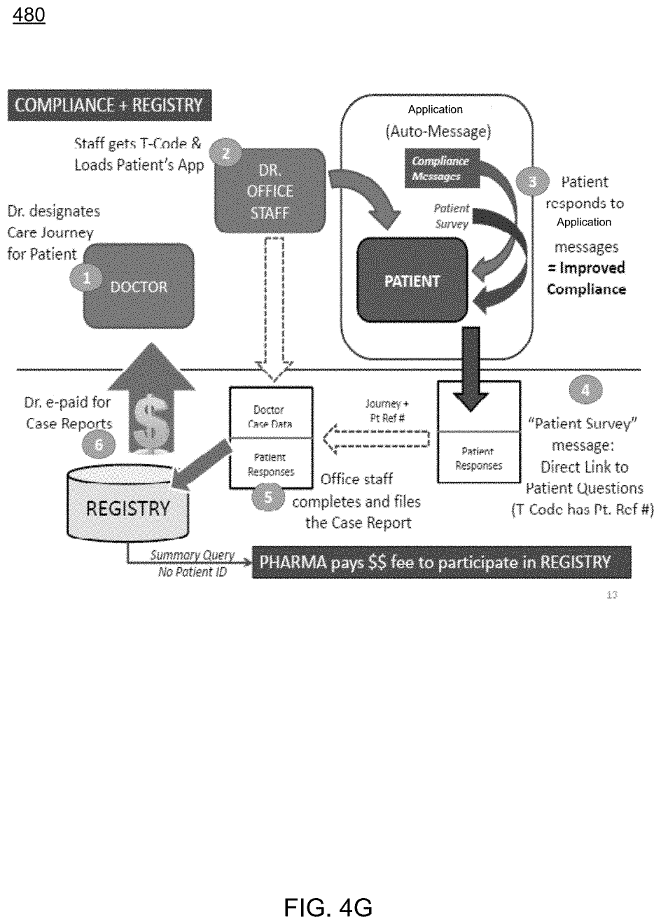

FIG. 4A illustrates an example setup configuration for a new course of treatment according to example embodiments. Referring to FIG. 4A, the illustration 400 includes the basic setup functions of linking a particular journey T-code (unique code) to the message and/or universal resource link (URL) to link the application of the user to a customized template, such as a response form, questionnaire, etc. The T-code, date, time, response, and other records for each instance may be stored in a patient record managed by the application system database. A T-code means return to provider (RTP) which indicates that a claim has reached its final disposition with no reimbursement due to billing errors. At the bottom of 4A an App ID is the applicant identification, the OVDT is the office visit date and time, PR # is the patient reply number and JID is the journey identification number. The database uses these identifiers to identify patients and isolate which forms to utilize. The form is dynamically filled out in the cloud server 245 with information from the patient records 250.

FIG. 4B illustrates an example database entry for the new course of treatment according to example embodiments. Referring to FIG. 4B, the example configuration 430 includes a database entry of messages which are organized by a category, in this case ophthalmology, and with a message content, including a link to a response page. The context and add-ons of a particular message may be customized based on a preferred layout or a default layout. The messages may be categorized in multiple ways by the cloud server, by patient, by journey, by response, by treatment, by drug, by drug interaction, and the like, in this way interactions of various kinds may be grouped by the system to potentially find commonalities, similarities, beneficial effects, side effects, etc. The response page may be similarly chosen by the cloud server based upon the initial journey or one of the groupings. The search by the cloud based server for similarities and interactions may allow the system to continuously learn from the patients what is the most favorable treatment and what common issues arrive and how those issues are dealt with for other patients having similar demographics and responses. The healthcare professional may also customize the interaction with the patient and the response of the system to certain criteria which he indicates.

FIG. 4C illustrates a flow diagram 440 configuration for the new course of treatment according to example embodiments. Referring to FIG. 4C, the flow diagram includes a daily batch of messages which are set up to be delivered to one or more devices associated with one or more assigned patients. The process begins with a trigger to send a message, such as a matured date or time. The process then continues to deliver additional messages once confirmation of delivery is made. If the message is delayed or the response required is not received, the message may be resent as a late message requiring immediate attention. The process may continue to cycle to identify whether any messages are outstanding or have not been confirmed. The system may track information here related to the type of illness/issue, treatment program, monitoring/effectiveness of a patient and tag according to responses and non-responses of the patient. The system may change tactics if the patient is unresponsive or answers responses indicating issues associated with the treatment journey. The system may change what questions are asked or the frequency of messaging based on the patient response or non-response. In the situation in which a patient is not responding, the system may have the healthcare provider initiate a call directly to the patient.

FIG. 4D illustrates an example list of messages for the ongoing course of treatment according to example embodiments. Referring to FIG. 4D, in this illustration 450, the various messages intended for a particular patient are illustrated by date. The messages may originate at the cloud based system and may be originally designed into the specific journey. The timing and response criteria may be set by the journey and may be modified by the healthcare professional.

FIG. 4E illustrates an example setup configuration for various courses of treatment according to example embodiments. Referring to FIG. 4E, the configuration 460 includes a menu of options along with a set of potential journeys the user may be assigned to manage the ongoing health care treatment plans for that user. The overview of treatment options and dates are included for reference purposes. The patient may be interacting with the system for multiple journeys simultaneously. Each journey may interact with the patient according to its own logical construct. The messages to be sent may originate at the cloud based server and the data received may be received by both the cloud based server and the healthcare provider server. In the cloud server the data is sorted according to patient demographics and interactions, in the healthcare provider server the data is sorted according to patient and healthcare provider. The logic governing the interactions may be controlled by the cloud based server and may be initially set by the specific journey.

FIG. 4F illustrates an example set of details of an ongoing course of treatment according to example embodiments. Referring to FIG. 4F, the details of the administrator are shown to include a journey builder function based on certain parameters, such as an identification code, specialty, a number and a sender name. The number of messages, responses and actions are recorded to demonstrate the user's interaction with the application and the specific treatment plan(s). The journey builder utilizes a spreadsheet style input that has a day, the message to send to the patient, the expected response and a link for the patient to link to. The system tracks the interactions between the sent messages and the patient responses. The patient device is tracked by a device universal identification (UID) and the application and menu items, the selection tiles and the T-codes. The system tracks and reports the number of specific messages sent to the patient, the number of clicks the patient performs and records the device T-code with each click in association with the patient identification number (Pt ID).

FIG. 4G illustrates an example network configuration of the various third parties involved in the application operation and compliance according to example embodiments. Referring to FIG. 4G, the network of communications among the integrated platform 480 demonstrates the process initiating with the doctor's office establishing a journey for the patient and assigning a T-code. The patient's responses are identified along with links and references to third party message links and other information sources. The doctor designates a journey for the patient based on the patient's condition and the treatment plan. The healthcare providers staff gets a T-code for the treatment plan and sends the application to the patients device. The journey application sends messages to the patient, receives responses to surveys and assesses compliance to the journey protocols. The patient responses to the patent survey messages directly link the response to the query for that patient. The journey sends the data to the cloud server in scrubbed form and sends the data to the healthcare providers server linking the patient reference number to the journey. The doctor's case files and the patient responses are compiled into a case report that is sent to a registry.

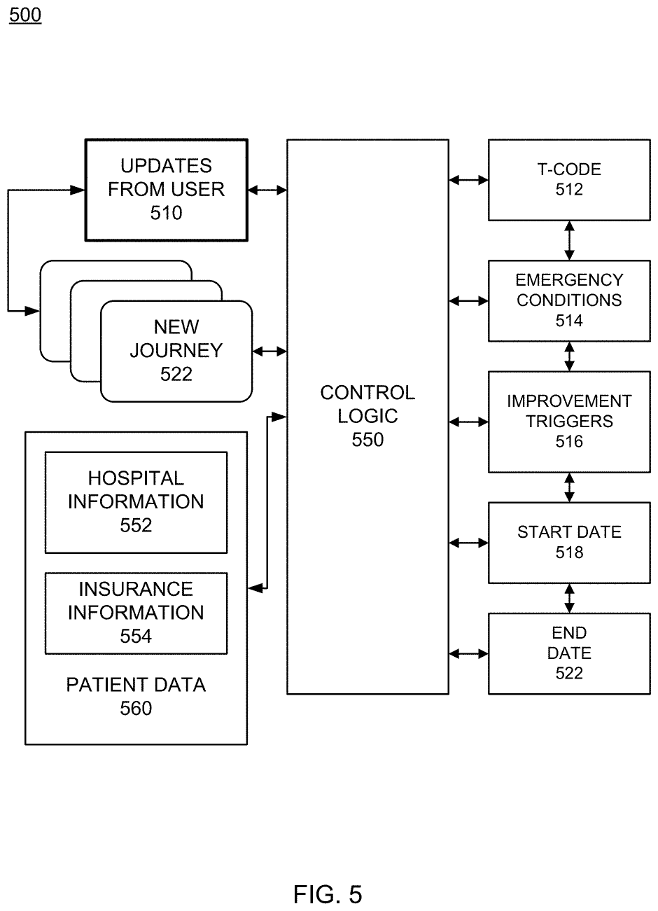

FIG. 5 illustrates a logic module configured to process the input and output parameters of the instant application according to example embodiments. Referring to FIG. 5, the control logic platform 500 includes a control logic unit 550, such as a processor or other processing entity that may receive updates from a user 510 such as by keyboard, voice processing and the like, new journey information 522 and/or patient data 560 including hospital 552, insurance 554, doctor, office, prescriptions and the like. The logic may be configured to identify and link the assigned T-code 512, emergency conditions 514, improvement triggers 516 for optimal changes to the treatment plan, along with dates 518 and new journey information 522 to perform the treatment plan.

In addition, while the term “message” has been used in the description of embodiments of the present application, the application may be applied to many types of network data, such as, packet, frame, datagram, etc. For purposes of this application, the term “message” also includes packet, frame, datagram, and any equivalents thereof. Furthermore, while certain types of messages and signaling are depicted in exemplary embodiments of the application, the application is not limited to a certain type of message, and the application is not limited to a certain type of signaling.

According to example embodiments, a user device, such as a smartphone, cellular phone, tablet device, laptop or other computing device with a memory and processor, may communicate with another computing device and/or a server to provide an integrated communication platform. A patient device as stated herein is a user device.

Example embodiments provide a computer system programmed to use automated messaging from medical offices to specific patients. The application is not limited to medical procedures and functions and may be used with other configurations for various purposes and services benefitting the end user. Example embodiments include three main computer systems, which work together in an integrated manner including a management platform that controls set-up, functionality, activity reporting, and messaging credentials for the users. An administrative platform which the doctor and doctor's office can access via the internet, and a mobile application that a patient can download into a mobile computer device such as a smartphone or tablet.

The integrated platform provides a way of checking-in with a patient at prescribed intervals during times between office visits and when undergoing certain treatment that the doctor is providing or overseeing for the patient. The patient dialog may gather relevant information about the status of the patient's conditions or recovery and can be modified or tailored to specifically meet the dialog requirements of the treating physician. Once initiated by the doctor's office, the application operates in an autonomous manner by delivering messages to the patient to prompt responses if needed. The application functions are monitored to assure that the patient replies to the information requests from the doctor, otherwise a no-response alert is sent to the doctor's office. The interactions are recorded and time-stamped, providing an auditable record of the dialog, suitable for insurance billing purposes. The application can also support parametric information from devices that measure certain body functions, such as diabetes glucometers, or blood pressure cuffs, or any sensory readable health care metric. The application may also create a longitudinal record of information for the patient to illustrate week-to-week trends.

The operations of a method or algorithm described in connection with the embodiments disclosed herein may be embodied directly in hardware, in a computer program executed by a processor, or in a combination of the two. A computer program may be embodied on a computer readable medium, such as a storage medium. For example, a computer program may reside in random access memory (“RAM”), flash memory, read-only memory (“ROM”), erasable programmable read-only memory (“EPROM”), electrically erasable programmable read-only memory (“EEPROM”), registers, hard disk, a removable disk, a compact disk read-only memory (“CD-ROM”), or any other form of storage medium known in the art.

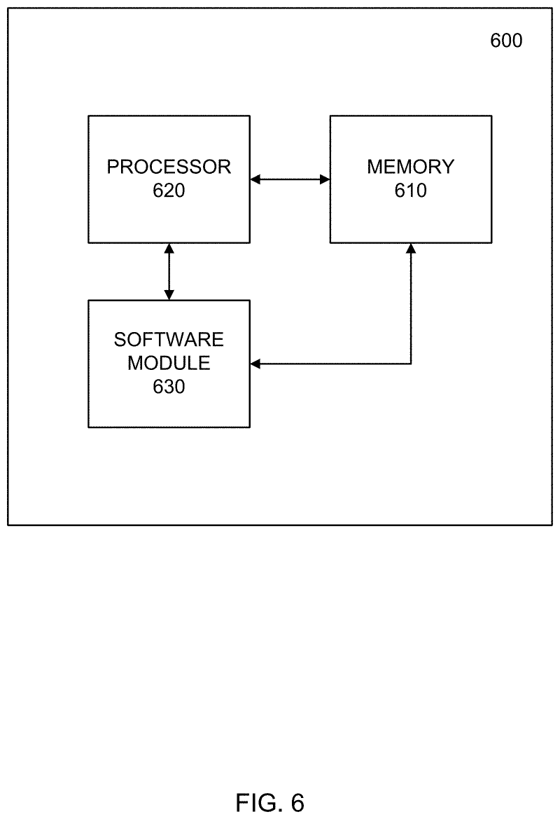

An exemplary storage medium may be coupled to the processor such that the processor may read information from, and write information to, the storage medium. In the alternative, the storage medium may be integral to the processor. The processor and the storage medium may reside in an application specific integrated circuit (“ASIC”). In the alternative, the processor and the storage medium may reside as discrete components. For example, FIG. 6 illustrates an example network entity 600, which may represent any of the above-described network components of the other figures.

As illustrated in FIG. 6, a memory 610 and a processor 620 may be discrete components of the network entity 600 that are used to execute an application or set of operations. The application may be coded in software in a computer language understood by the processor 620, and stored in a computer readable medium, such as, the memory 610. The computer readable medium may be a non-transitory computer readable medium that includes tangible hardware components in addition to software stored in memory. Furthermore, a software module 630 may be another discrete entity that is part of the network entity 600, and which contains software instructions that may be executed by the processor 620. In addition to the above noted components of the network entity 600, the network entity 600 may also have a transmitter and receiver pair configured to receive and transmit communication signals (not shown).

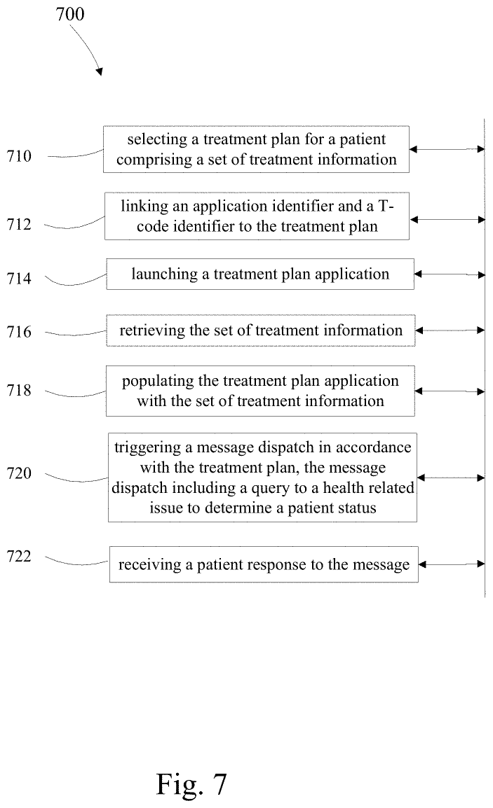

A first example method shown in FIG. 7, 700, may include, selecting 710 a treatment plan for a patient comprising a set of treatment information, linking 712 an application identifier and a T-code identifier to the treatment plan and launching 714 a treatment plan application. The method further includes retrieving 716 the set of treatment information, populating 718 the treatment plan application with the set of treatment information and triggering 720 a message dispatch in accordance with the treatment plan. The message dispatch includes a query to a health related issue to determine a patient status and the system receives 722 a patient response to the message.

Communication Platform Builder

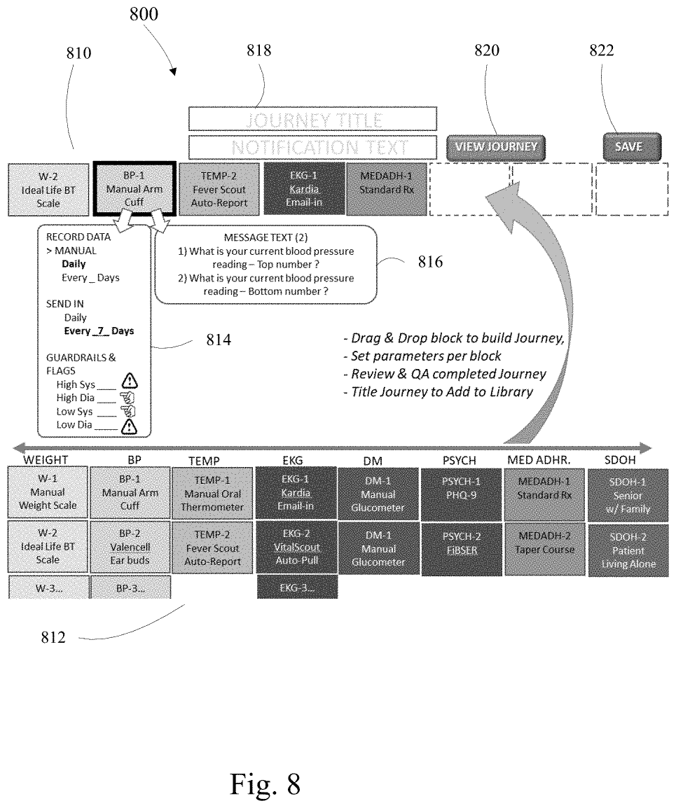

One example screen layout of the communication platform builder is depicted in FIG. 8, wherein the treatment plan table has a top portion 810 and a bottom portion 812. The top portion 810 is the specific treatment plan for a patient that is built from the selectable parameter blocks from the bottom portion. The screen depicted in this example is on a healthcare provider's screen, the data is provided by the cloud based server, the initial guardrails and flags are set by the cloud based server, but may be modified by the healthcare provider.

The selectable parameter blocks are broken into block subsets such as weight, blood pressure, temperature, electro-cardiogram, diabetes monitoring, psychological monitoring, medication adherence and habitat monitoring. Within each of the block subsets there are associated selectable parameter blocks. The blocks are provided by the cloud based server and may be chosen by the healthcare provider to modify a journey or create a journey.

The weight block subset may have a manual weight input parameter block, connection weight input parameter block and the like. The blood pressure block subset may have manual arm pressure cuff input parameter block, an ear bud parameter block and the like. The temperature block subset may have a manual oral temperature parameter block, a fever scout auto-report parameter block and the like. The electrocardiogram block subset may have a specific manufacturer email in parameter block, a wearable patch that provides ECG data, auto-pull parameter block and the like. The diabetes monitoring block subset may have different manual glucometer parameter blocks and wearable glucose monitoring parameter block inputs and the like. The psychological monitoring block subset may have a standardized patient health questionnaire, a frequency, intensity, burden of side effects (FiBSER) parameter block and the like. The medical adherence monitoring block may have a standard medication parameter block and a taper course parameter block. The habitat monitor block may have a senior living with family, a patient living alone parameter block and the like.

As an example, within the blood pressure subset 814, manual arm pressure cuff input parameter block, there may be a recording interval within which the data is to be recorded, the recording may be manual, or if an automatic arm pressure cuff with communication capabilities is utilized, the data may be uploaded directly from the device, a communication interval within which the data is communicated to the health care provider through the system by messages sent from the cloud based server to the healthcare provider and a set of guardrails and flags indicating low and high blood pressure values. The manual arm pressure cuff input parameter block may also include text messages 816 requesting specific information from the patient.

The various input parameter blocks may be suggested by the cloud based system and from these, specific blocks dragged and dropped into a specific treatment plan. A title identifier 818 may be input to the specific treatment plan to be added to a library of treatment plans. Once the various input parameter blocks are placed into the treatment plan a review and quality control may be performed on the treatment plan to insure that each relevant indices for the patient is addressed. The quality control may be performed by the cloud based system reviewing the plan for errors or substantial differences from similar treatment plans. The treatment plan table may be viewed 820 and saved 822.

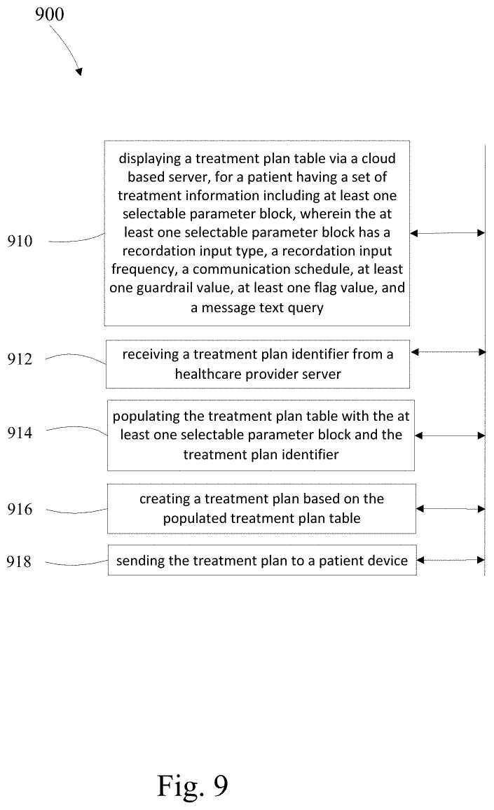

In an example depicted in FIG. 9, a method, comprising displaying 910 a treatment plan table via a cloud based server, for a patient having a set of treatment information including at least one selectable parameter block, wherein the at least one selectable parameter block has a recordation input type, a recordation input frequency, a communication schedule, at least one guardrail value indicating a value that is non-normal for the patient, at least one flag value, and a message text query, receiving 912 a treatment plan identifier from a healthcare provider server, populating 914 the treatment plan table with the at least one selectable parameter block and the treatment plan identifier, creating 916 a treatment plan based on the populated treatment plan table and sending 918 the treatment plan to a patient device.

The cloud based server as discussed herein is a central repository of data to be utilized by a healthcare provider server. The cloud based server may assess the original treatment in light of a pool of similar patients to the current patient, based on condition, age, sex, demographics and the like and may propose an alternative treatment based on a positive outcome for the similar pool of patients. The treatments for the pool of patients and their outcomes may be stored on the cloud server in a format which does not identify the particular patients of the pool, but does describe their characteristics and outcomes. The data from the cloud server may be scrubbed and may be disease, patient characteristic, regimen and outcome centric. This collection and correlation of data may allow for continuous improvement in treatment proposals to the healthcare provider.

The method may include loading a trigger table having a set of trigger dates based on the treatment plan, a message dispatch responsive to the set of trigger dates. The selectable parameter block may be drag and drop, where parameters are individually selectable within the selectable parameter block. The method may include displaying a review display to review a set of selected parameter blocks and review parameters within the set of selected parameter blocks and displaying a quality assurance display that detects whether additional components should be added based on similar treatment plans. The treatment plan may be added to a treatment plan library. The recordation input type is of manual and wirelessly received receiving parametric device information of patient body functions. The method may include storing a T-code identifier, a timestamp and a patient response to an auditable patient record and the treatment plan may be modified.

In another example, a non-transitory computer readable medium comprising instructions that, when read by a processor, cause the processor to perform, displaying a treatment plan table via a cloud based server, for a patient having a set of treatment information including at least one selectable parameter block, wherein the at least one selectable parameter block has a recordation input type, a recordation input frequency, a communication schedule, at least one guardrail value, at least one flag value, and a message text query. The instructions further comprise receiving a treatment plan identifier from a healthcare provider server, populating the treatment plan table with the at least one selectable parameter block and the treatment plan identifier, creating a treatment plan based on the populated treatment plan table and sending the treatment plan to a patient device.

In yet a further example, a cloud-based processor, and memory electrically coupled to the cloud-based processor and storing an application where the cloud-based processor performs operations to, display a treatment plan table via a cloud based server, for a patient having a set of treatment information including at least one selectable parameter block, wherein the at least one selectable parameter block has a recordation input type, a recordation input frequency, a communication schedule, at least one guardrail value, at least one flag value, and a message text query. The instructions further cause the processor to receive a treatment plan identifier from a healthcare provider server, populate the treatment plan table with the at least one selectable parameter block and the treatment plan identifier, create a treatment plan based on the populated treatment plan table and send the treatment plan to a patient device.

With respect to the timing of patient responses, the first example method may also include, awaiting the patient response to the message for a late response duration and categorizing the patient response if the patient response is received within the late response duration. If the patient response is not received within the late response duration the method further comprises sending a duplicate message and flagging the patient response as non-responsive if the patient response to the duplicate message is not received within a second late response duration.

The timing of the message dispatches associated with the treatment plan is partly governed by a trigger table. The method may include loading the trigger table having a set of trigger dates based on the treatment plan where the message dispatch is sent according to the set of trigger dates. The method may further include receiving a message start date and receiving an initialization message from a patient mobile device to initiate the treatment plan and to initialize the set of trigger dates in the trigger table.

Although an exemplary embodiment of the system, method, and computer readable medium of the present application has been illustrated in the accompanied drawings and described in the foregoing detailed description, it will be understood that the application is not limited to the embodiments disclosed, but is capable of numerous rearrangements, modifications, and substitutions without departing from the spirit or scope of the application as set forth and defined by the following claims. For example, the capabilities of the system of the various figures can be performed by one or more of the modules or components described herein or in a distributed architecture and may include a transmitter, receiver or pair of both. For example, all or part of the functionality performed by the individual modules, may be performed by one or more of these modules. Further, the functionality described herein may be performed at various times and in relation to various events, internal or external to the modules or components. Also, the information sent between various modules can be sent between the modules via at least one of: a data network, the Internet, a voice network, an Internet Protocol network, a wireless device, a wired device and/or via plurality of protocols. Also, the messages sent or received by any of the modules may be sent or received directly and/or via one or more of the other modules.

One skilled in the art will appreciate that a “system” could be embodied as a personal computer, a server, a console, a personal digital assistant (PDA), a cell phone, a tablet computing device, a smartphone or any other suitable computing device, or combination of devices. Presenting the above-described functions as being performed by a “system” is not intended to limit the scope of the present application in any way, but is intended to provide one example of many embodiments of the present application. Indeed, methods, systems and apparatuses disclosed herein may be implemented in localized and distributed forms consistent with computing technology.

It should be noted that some of the system features described in this specification have been presented as modules, in order to more particularly emphasize their implementation independence. For example, a module may be implemented as a hardware circuit comprising custom very large scale integration (VLSI) circuits or gate arrays, off-the-shelf semiconductors such as logic chips, transistors, or other discrete components. A module may also be implemented in programmable hardware devices such as field programmable gate arrays, programmable array logic, programmable logic devices, graphics processing units, or the like.

A module may also be at least partially implemented in software for execution by various types of processors. An identified unit of executable code may, for instance, comprise one or more physical or logical blocks of computer instructions that may, for instance, be organized as an object, procedure, or function. Nevertheless, the executables of an identified module need not be physically located together, but may comprise disparate instructions stored in different locations which, when joined logically together, comprise the module and achieve the stated purpose for the module. Further, modules may be stored on a computer-readable medium, which may be, for instance, a hard disk drive, flash device, random access memory (RAM), tape, or any other such medium used to store data.

Indeed, a module of executable code could be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices, and may exist, at least partially, merely as electronic signals on a system or network.

It will be readily understood that the components of the application, as generally described and illustrated in the figures herein, may be arranged and designed in a wide variety of different configurations. Thus, the detailed description of the embodiments is not intended to limit the scope of the application as claimed, but is merely representative of selected embodiments of the application.

One having ordinary skill in the art will readily understand that the application as discussed above may be practiced with steps in a different order, and/or with hardware elements in configurations that are different than those which are disclosed. Therefore, although the application has been described based upon these preferred embodiments, it would be apparent to those of skill in the art that certain modifications, variations, and alternative constructions would be apparent, while remaining within the spirit and scope of the application. In order to determine the metes and bounds of the application, therefore, reference should be made to the appended claims.

While preferred embodiments of the present application have been described, it is to be understood that the embodiments described are illustrative only and the scope of the application is to be defined solely by the appended claims when considered with a full range of equivalents and modifications (e.g., protocols, hardware devices, software platforms etc.) thereto.

What is claimed is:

1. A method, comprising:
displaying a treatment plan table via a cloud based server, for a patient having a set of treatment information including at least one selectable parameter block, wherein the at least one selectable parameter block has a recordation input type, a recordation input frequency, a communication schedule, at least one guardrail value, at least one flag value, and a message text query;
receiving a treatment plan identifier from a healthcare provider server;
populating the treatment plan table with the at least one selectable parameter block and the treatment plan identifier;
creating a treatment plan based on the populated treatment plan table; and
sending the treatment plan to a patient device.

2. The method of claim 1, further comprising loading a trigger table having a set of trigger dates based on the treatment plan, a message dispatch responsive to the set of trigger dates.

3. The method of claim 1, wherein the at least one selectable parameter block is drag and drop.

4. The method of claim 1, wherein parameters are individually selectable within the at least one selectable parameter block.

5. The method of claim 4, further comprising displaying a review display to review a set of selected parameter blocks and review parameters within the set of selected parameter blocks.

6. The method of claim 5, further comprising displaying a quality assurance display that detects whether additional components should be added based on similar treatment plans.

7. The method of claim 1, further comprising adding the treatment plan to a treatment plan library.

8. The method of claim 1, wherein the recordation input type is at least one of manual and wirelessly received receiving parametric device information of patient body functions.

9. The method of claim 1, further comprising storing a T-code identifier, a timestamp and a patient response to an auditable patient record.

10. The method of claim 1, further comprising modifying the treatment plan.

11. A non-transitory computer readable medium comprising instructions that, when read by a processor, cause the processor to perform:
displaying a treatment plan table via a cloud based server, for a patient having a set of treatment information including at least one selectable parameter block, wherein the at least one selectable parameter block has a recordation input type, a recordation input frequency, a communication schedule, at least one guardrail value, at least one flag value, and a message text query;
receiving a treatment plan identifier from a healthcare provider server;
populating the treatment plan table with the at least one selectable parameter block and the treatment plan identifier;
creating a treatment plan based on the populated treatment plan table; and
sending the treatment plan to a patient device.

12. The non-transitory computer readable medium of claim 11, comprising instructions, that when read by a processor, cause the processor to perform loading a trigger table having a set of trigger dates based on the treatment plan, a message dispatch responsive to the set of trigger dates.

13. The non-transitory computer readable medium of claim 11, wherein the at least one selectable parameter block is drag and drop.

14. The non-transitory computer readable medium of claim 11, wherein parameters are individually selectable within the at least one selectable parameter block.

15. The non-transitory computer readable medium of claim 14, comprising instructions, that when read by a processor, cause the processor to perform displaying a review display to review a set of selected parameter blocks and review parameters within the set of selected parameter blocks.

16. The non-transitory computer readable medium of claim 15, comprising instructions, that when read by a processor, cause the processor to perform displaying a quality assurance display that detects whether additional components should be added based on similar treatment plans.

17. The non-transitory computer readable medium of claim 11, comprising instructions, that when read by a processor, cause the processor to perform adding the treatment plan to a treatment plan library.

18. The non-transitory computer readable medium of claim 11, wherein the recordation input type is at least one of manual and wirelessly received receiving parametric device information of patient body functions.

19. A system, comprising:
at least one cloud-based processor; and
at least one memory electrically coupled to the at least one cloud-based processor and storing an application, wherein the at least one cloud-based processor performs operations to:
display a treatment plan table via a cloud based server, for a patient having a set of treatment information including at least one selectable parameter block, wherein the at least one selectable parameter block has a recordation input type, a recordation input frequency, a communication schedule, at least one guardrail value, at least one flag value, and a message text query;
receive a treatment plan identifier from a healthcare provider server;
populate the treatment plan table with the at least one selectable parameter block and the treatment plan identifier;
create a treatment plan based on the populated treatment plan table; and
send the treatment plan to a patient device.

20. The system of claim 19, wherein the at least one cloud-based processor further performs an operation to load a trigger table having a set of trigger dates based on the treatment plan, a message dispatch responsive to the set of trigger dates.
